(19) United States
(12) Patent Application Publication
(10) Pub. No.: US 2023/0225889 A1
(43) Pub. Date: Jul. 20, 2023

(54) STENT, STENT DELIVERY SYSTEM, AND STENT MANUFACTURING METHOD

(71) Applicant: OLMPUS CORPORATION, Tokyo (JP)

(72) Inventor: Shun NOGUCHI, Tokyo (JP)

(21) Appl. No.: 18/183,636

(22) Filed: Mar. 14, 2023

Related U.S. Application Data
(63) Continuation of application No. PCT/JP2020/036759, filed on Sep. 29, 2020.

(51) Int. Cl. A61F 2/90

(52) U.S. Cl. CPC A61F 2/90; A61F 2220/0091

(57) ABSTRACT

A tubular stent formed of at least one wire includes proximal and distal ends and an intermediate part that includes annular windings extending in a circumferential direction around a longitudinal axis of the stent and arranged in a direction along the longitudinal axis. Each of the windings has hill-shaped sections bent toward the proximal end and valley-shaped sections bent toward the distal end, alternately arranged in the circumferential direction. All sections form intersections helically arranged around the longitudinal axis. The hill-shaped sections of one of the windings and the valley-shaped sections of the neighboring winding overlap each other at the intersections in a radial direction of the stent. Distances in the direction of the longitudinal axis between the hill-shaped and the valley-shaped sections of a first winding forming at least one of the proximal and distal ends are different from each other in a circumferential direction of the stent.

### TECHNICAL FIELD

This is a continuation of International Application PCT/JP2020/036759 which is hereby incorporated by reference herein in its entirety

The present invention relates to stents, stent delivery systems, and stent manufacturing methods.

### BACKGROUND ART

A known stent in the related art is disposed in a narrow area of a tubular organ, such as a blood vessel, the trachea, a bile duct, the esophagus, the duodenum, or the urethra, and expands the narrow area (e.g., see Patent Literatures 1 and 2). In order to reduce the load on the narrow area, it is desirable that the stent have flexibility high enough to maintain its shape in conformity to the shape of the narrow area regardless of whether the narrow area is linear or bent. A stent with low flexibility generates an elastic force to restore its linear shape in a bent narrow area, possibly applying load on the narrow area as a result of the opposite ends of the stent coming into contact with the inner wall of the narrow area with a strong force.

A stent according to Patent Literature 1 has intertwined sections where a plurality of zigzag sections of a wire are intertwined with each other. The intertwined sections give flexibility to the stent.

A stent according to Patent Literature 2 has a plurality of zigzag-shaped linear helical members extending parallel to each other.

### CITATION LIST

### Patent Literature

### PTL 1

### The Publication of Japanese Patent No. 4451421

### PTL 2

### Japanese Unexamined Patent Application, Publication No. 2003-102849

### SUMMARY OF INVENTION

An aspect of the present invention provides a tubular stent formed of at least one wire. The stent includes a proximal end, a distal end, and an intermediate part interposed between the proximal end and the distal end. The intermediate part includes a plurality of annular windings extending in a circumferential direction around a longitudinal axis of the stent and arranged in a direction along the longitudinal axis. Each of the plurality of windings has a zigzag shape with hill-shaped sections and valley-shaped sections alternately arranged in the circumferential direction, each of the hill-shaped sections being a part of each of the plurality of windings bent toward the proximal end, each of the valley-shaped sections being a part of each of the plurality windings bent toward the distal end. All the hill-shaped sections and all the valley-shaped sections form intersections, the hill-shaped sections of one of the plurality of windings and the valley-shaped sections of another one of the plurality of windings adjacent to the one of the plurality of windings respectively overlapping each other at the intersections in a radial direction of the stent. The intersections are helically arranged around the longitudinal axis. Distances in the direction of the longitudinal axis between the hill-shaped sections and the valley-shaped sections of a first winding of the plurality of windings are different from each other in a circumferential direction of the stent, the hill-shaped sections and the valley-shaped sections of the first winding forming at least one of the proximal end and the distal end.

### BRIEF DESCRIPTION OF DRAWINGS

FIG. 1 schematically illustrates the overall configuration of a stent according to an embodiment of the present invention.

FIG. 2A is a partially enlarged view of the stent in FIG. 1.

FIG. 2B is another partially enlarged view of the stent in FIG. 1.

FIG. 3 illustrates an example of an unfolded view of the stent in FIG. 1.

FIG. 4A illustrates an intertwined section of a first pattern.

FIG. 4B illustrates an intertwined section of a second pattern.

FIG. 5A illustrates a non-intertwined section of a first pattern.

FIG. 5B illustrates a non-intertwined section of a second pattern.

FIG. 6 illustrates the overall configuration of a stent delivery system according to an embodiment of the present invention.

FIG. 7A is a vertical sectional view of a distal end of the stent delivery system in FIG. 6.

FIG. 7B is a side view of the distal end of the stent delivery system in FIG. 6 and illustrates the operation of a delivery catheter for releasing the stent.

FIG. 8 is a graph illustrating the relationship between a bending angle and an amount of force in the stent according to the present invention and a stent according to a comparative example.

FIG. 9 is a graph illustrating the relationship between the bending angle and an elastic force in the stent according to the present invention and the stent according to the comparative example.

FIG. 10 illustrates a method for manufacturing the stent in FIG. 1.

FIG. 11A is a partially enlarged view of a proximal end in a modification of the stent in FIG. 1.

FIG. 11B is a partially enlarged view of the proximal end in another modification of the stent in FIG. 1.

FIG. 12 is a partially enlarged view of the proximal end in another modification of the stent in FIG. 1.

FIG. 13 illustrates an example where the stent in FIG. 1 is applied to a lower bile duct.

FIG. 14 is a partially enlarged view illustrating an end treatment performed on the stent in FIG. 1.

FIG. 15 schematically illustrates the overall configuration of a modification of the stent in FIG. 1.

### DESCRIPTION OF EMBODIMENTS

A stent and a stent delivery system according to an embodiment of the present invention will be described below with reference to the drawings.

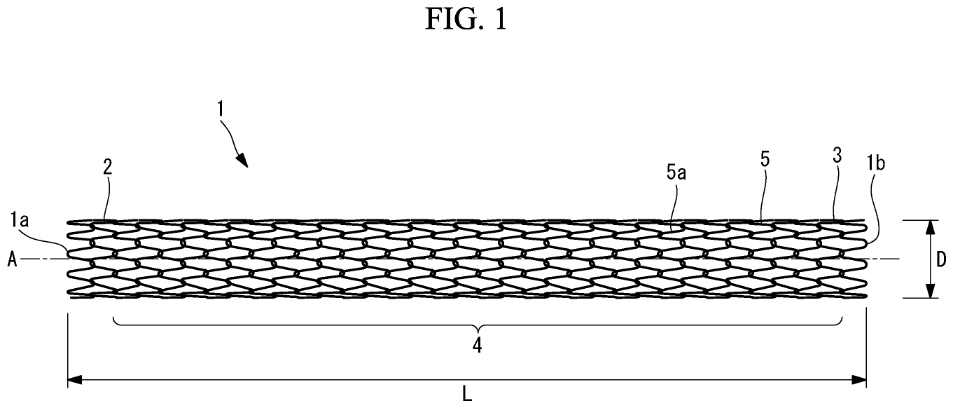

As shown in FIG. 1, a stent 1 according to this embodiment has a cylindrical shape with openings at the opposite ends thereof, and has a linear original shape with a predetermined diameter in a natural state where an external force is not applied to the stent 1. The stent 1 includes a proximal end 2 and a distal end 3 that are located at the opposite longitudinal ends of the stent 1, and a long intermediate part 4 interposed between the proximal end 2 and the distal end 3.

The stent 1 is formed of a single wire 5 alone. For example, the wire 5 has a diameter ranging between 0.1 mm and 0.5 mm and is composed of a shape-memory alloy, such as a nickel-titanium alloy. The stent 1 has a mesh pattern with multiple rhombic meshes arranged in the circumferential direction and the longitudinal direction, and is manufactured by weaving the single wire 5, as will be described below. The stent 1 contracts in the radial direction in accordance with a radially-inward external force, and self-expands in the radial direction by releasing the external force.

The wire 5 is helically wound multiple times around a longitudinal axis A of the stent 1, so that a plurality of open-circular windings 5a extending circumferentially around the longitudinal axis A and arranged in the direction along the longitudinal axis A are formed. A proximal end and a distal end of the wire 5 are joined to other parts of the wire 5. The intermediate part 4 is constituted of a plurality of the windings 5a. Each winding 5a has a plurality of hill-shaped sections 6 and a plurality of valley-shaped sections 7. The hill-shaped sections 6 and the valley-shaped sections 7 are alternately arranged in the circumferential direction around the longitudinal axis A to form a zigzag shape (e.g., a triangular wave shape or a sinusoidal shape). Each hill-shaped section 6 is a part of the winding 5a bent toward the proximal end 2 and protruding toward the distal end 3. Each valley-shaped sections 7 is a part of the winding 5a bent toward the distal end 3 and protruding toward the proximal end 2.

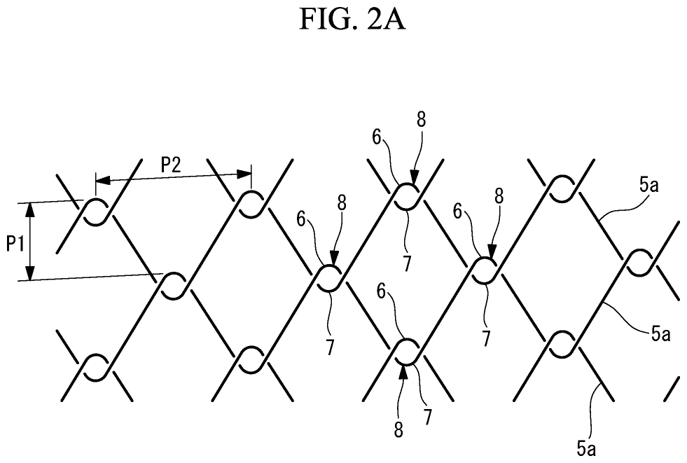

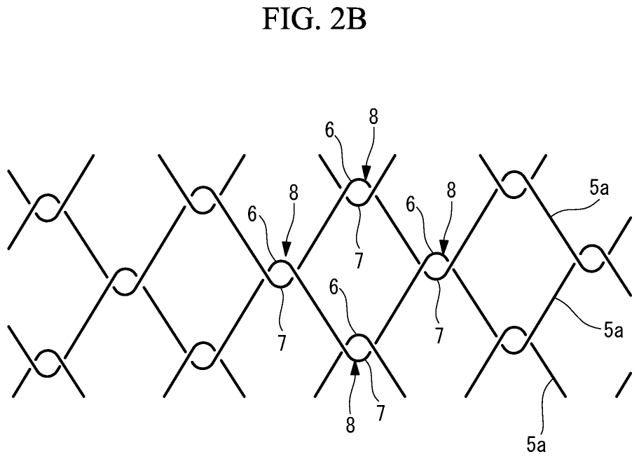

As shown in FIGS. 1 and 2A, design values of the stent 1 include a length L, a diameter D, a pitch P1 of the helix, and a pitch P2 of the hill-shaped sections 6. The pitch P1 is the distance between two neighboring windings 5a in the direction along the longitudinal axis A. The pitch P2 is the distance, in the circumferential direction, between two hill-shaped sections 6 adjacent to each other in the circumferential direction, and is determined in accordance with the number of hill-shaped sections 6 per single winding 5a. The stent 1 according to this embodiment may be applied to any of various tubular organs, such as a blood vessel, the trachea, a bile duct, the esophagus, the duodenum, or the urethra. The design values are set in accordance with an application site of the stent 1.

For example, in a case where the stent 1 is for a bile duct, the length L ranges between 20 mm and 200 mm, the diameter D ranges between 4 mm and 15 mm, the pitch P1 of the helix ranges between 1 mm and 5 mm, and the number of hill-shaped sections 6 per single winding 5a ranges between 7.5 and 13.5.

As shown in FIGS. 1, 2A, and 2B, an intersection 8 formed by a pair of hill-shaped section 6 and valley-shaped section 7 is located at each of the four apexes of each rhombic mesh. Each intersection 8 is where the hill-shaped section 6 of one winding 5a and the valley-shaped section 7 of another winding 5a adjacent to the distal side of the one winding 5a overlap each other in the radial direction of the stent 1. When each intersection 8 is viewed from the front, a part of the wire 5 serving as the hill-shaped section 6 and a part of the wire 5 serving as the valley-shaped section 7 intersect with each other at the intersection 8. Each winding 5a in the intermediate part 4 intersects with another winding 5a only at the intersections 8.

The intersections 8 are helically arranged around the longitudinal axis A. Specifically, the hill-shaped sections 6 are helically arranged at the pitch P2 around the longitudinal axis A. The hill-shaped section 6 of one winding 5a and the valley-shaped section 7 of another winding 5a adjacent to the distal side of the one winding 5a are disposed at the same position or substantially the same position in the circumferential direction around the longitudinal axis A. All the hill-shaped sections 6 and all the valley-shaped sections 7 form the intersections 8 in at least the intermediate part 4.

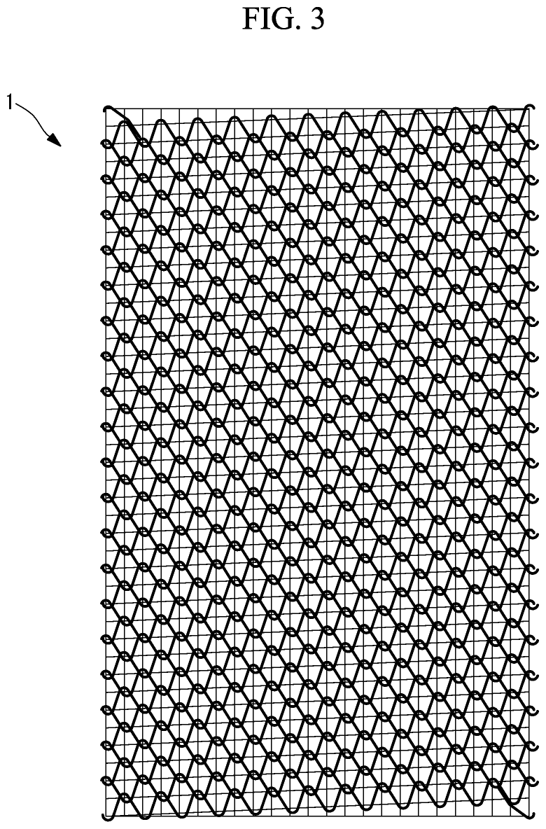

FIG. 3 is a development view of the stent 1. In FIG. 3, the lower side corresponds to the proximal side, the upper side corresponds to the distal side, and the left-right direction corresponds to the circumferential direction. As shown in FIG. 3, the wire 5 is bent at transition points serving as intersection points between a plurality of circumference division lines and length division lines, and have the hill-shaped sections 6, the valley-shaped sections 7, and the intersections 8 at the transition points. The circumference division lines extend in the longitudinal direction of the stent 1 and equally divide the circumference of the stent 1 into a plurality of segments. The length division lines extend in the circumferential direction of the stent 1 and equally divide the length of the stent 1 into a plurality of segments.

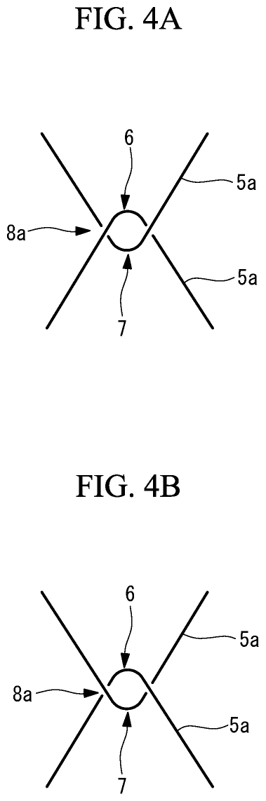

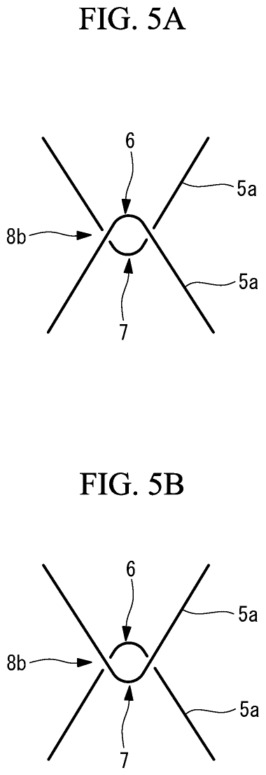

Each intersection 8 is one of two types, namely, an intertwined section 8a shown in FIGS. 4A and 4B and a non-intertwined section 8b shown in FIGS. 5A and 5B. Each intersection 8 in the intermediate part 4 is either one of the intertwined section 8a and the non-intertwined section 8b. At the intertwined section 8a, the part of the wire 5 serving as the hill-shaped section 6 and the part of the wire 5 serving as the valley-shaped section 7 intertwine with each other. At the non-intertwined section 8b, the hill-shaped section 6 and the valley-shaped section 7 are arranged in the radial direction of the stent 1 without the part of the wire 5 serving as the hill-shaped section 6 and the part of the wire 5 serving as the valley-shaped section 7 intertwining with each other, such that the entire hill-shaped section 6 is disposed radially outward or inward of the entire valley-shaped section 7.

Each winding 5a has at least one hill-shaped section 6 that forms an intertwined section 8a. All the intersections 8 in the intermediate part 4 may be intertwined sections 8a. Alternatively, some of the intersections 8 in the intermediate part 4 may be intertwined sections 8a, whereas the other intersections 8 in the intermediate part 4 may be non-intertwined sections 8b.

Two neighboring windings 5a are coupled to each other at the intertwined sections 8a, so that the stent 1 can maintain its cylindrical shape. It is preferable that each winding 5a be coupled to another neighboring winding 5a only at the intertwined sections 8a.

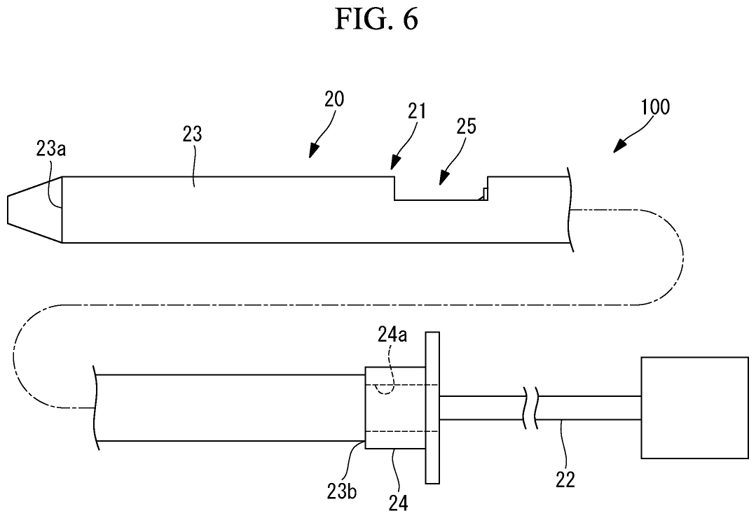

As shown in FIG. 6, a stent delivery system 100 according to this embodiment includes a long delivery catheter 20 and the stent 1, which is self-expandable.

The delivery catheter (delivery device) 20 includes a long tubular outer cylindrical unit (sheath) 21 and a long inner cylindrical unit 22 inserted in the outer cylindrical unit 21. The inner cylindrical unit 22 is movable relative to the outer cylindrical unit 21 in the longitudinal direction of the outer cylindrical unit 21.

The outer cylindrical unit 21 includes an outer cylinder 23 and a gripper 24 attached to one end of the outer cylinder 23.

The outer cylinder 23 is composed of, for example, resin and has flexibility. The outer cylinder 23 has openings at a distal end 23a and at a proximal end 23b. The openings communicate with the internal space (lumen) of the outer cylinder 23. The outer peripheral surface of the outer cylinder 23 in an intermediate area thereof in the longitudinal direction is provided with a side hole 25 that communicates with the lumen.

The gripper 24 is attached to the proximal end 23b of the outer cylinder 23. The gripper 24 has a through-hole 24a. The through-hole 24a communicates with the lumen of the outer cylinder 23. The shape of the gripper 24 is not particularly limited. The gripper 24 may be integrated with the outer cylinder 23 by, for example, resin molding.

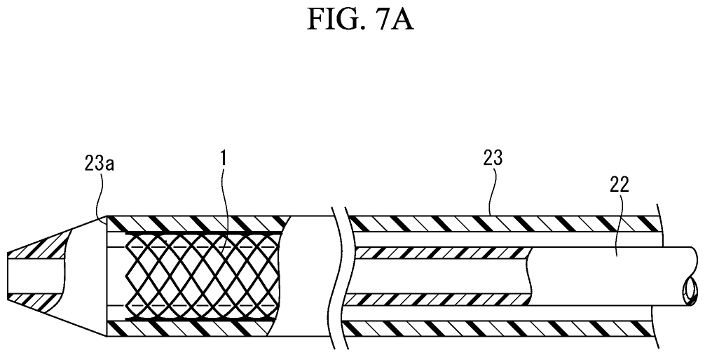

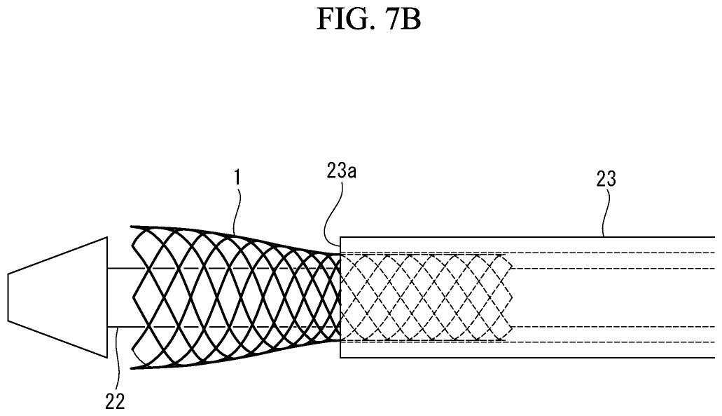

As shown in FIG. 7A, the stent 1 in a contracted state is fitted into the distal end of the delivery catheter 20. The delivery catheter 20 retains the stent 1 such that the stent 1 is released outward from the delivery catheter 20 in accordance with relative movement between the outer cylindrical unit 21 and the inner cylindrical unit 22 in the longitudinal direction of the outer cylindrical unit 21.

For example, the stent 1 is disposed in a contracted state within a cylindrical shape between the inner cylindrical unit 22 and the outer cylinder 23. The outer peripheral surface of the inner cylindrical unit 22 is provided with a stopper, such as a protrusion, protruding radially outward and inserted into a mesh of the stent 1. With the stopper, the stent 1 is attached to the distal end of the inner cylindrical unit 22 in such a manner as to be movable in the longitudinal direction of the outer cylindrical unit 21 together with the inner cylindrical unit 22 relative to the outer cylindrical unit 21. By extending the inner cylindrical unit 22 or by retracting the outer cylindrical unit 21, the distal end of the inner cylindrical unit 22 is caused to protrude from the distal end of the outer cylindrical unit 21, thereby releasing the stent 1.

Next, the operation of the stent 1 will be described. The stent 1 according to this embodiment is inserted into a narrow area of a tubular organ in the body by using the delivery catheter 20. The tubular organ is, for example, a bile duct.

After inserting the distal end of the delivery catheter 20 to a location near the narrow area, the stent 1 is released outward from the distal end of the outer cylinder 23 by causing the distal end of the inner cylindrical unit 22 to protrude from the distal end of the outer cylindrical unit 21, as shown in FIG. 7B. The stent 1 is disposed in the narrow area as a result of being released from the outer cylinder 23, and self-expands outward in the radial direction so as to expand the narrow area in the radial direction. FIG. 7B illustrates a state where the stent 1 is being released and is expanding. If the narrow area is curved or bent, the stent 1 bends in a direction intersecting the longitudinal direction of the stent 1 in accordance with a bending force received from the narrow area.

The stent 1 bends as a result of two neighboring windings 5a shifting relative to each other. The hill-shaped sections 6 and the valley-shaped sections 7 at the intertwined sections 8a where the two neighboring windings 5a are coupled to each other are smoothly shiftable relative to each other in accordance with the bending force applied to the stent 1. In detail, when the stent 1 bends, the hill-shaped sections 6 and the valley-shaped sections 7 at the intertwined sections 8a move relative to each other along the longitudinal axis A or rotate relative to each other via the intersections 8 around an axis intersecting the longitudinal axis A, depending on the location in the stent 1.

Specifically, when the stent 1 bends, deformation hardly occurs or does not occur at the parts of the windings 5a serving as the hill-shaped sections 6 and the valley-shaped sections 7 at the intertwined sections 8a, so that an elastic force that restores the stent 1 to a linear shape hardly occurs or does not occur at the intertwined sections 8a. Therefore, the stent 1 has flexibility high enough to bend with a small force, and once the stent 1 bends, the stent 1 maintains the same bent shape as the narrow area. Such a stent 1 can stay in the narrow area without applying load on the narrow area even if the narrow area is curved or bent.

Because the hill-shaped sections 6 and the valley-shaped sections 7 forming the non-intertwined sections 8b are not restrained by each other, the hill-shaped sections 6 and the valley-shaped sections 7 are freely shiftable when the stent 1 bends, so that the non-intertwined sections 8b do not contribute to an elastic force with respect to the deformation of the stent 1. Therefore, with some of the intersections 8 in the intermediate part 4 being the non-intertwined sections 8b, the elastic force can be further reduced when the stent 1 deforms, thereby further increasing the flexibility of the stent 1.

Joint sections where the ends of the wire 5 and other parts of the wire 5 are joined to each other may possibly generate an elastic force when the stent 1 bends. In this embodiment, the stent 1 is formed of a single wire 5 alone, so that the number of joint sections can be minimized.

With the intermediate part 4 being provided with the non-intertwined sections 8b, a stent-in-stent technique using two stents 1 is readily achievable. A stent-in-stent technique involves retaining two stents in a Y-shaped fashion by routing a second stent through the mesh of a first stent from the inside to the outside of the first stent. The stent-in-stent technique is used in a bifurcated tubular organ, such as a bile duct.

Four rhombic meshes surrounding a single non-intertwined section 8b form a single large mesh. Therefore, by routing the second stent through the single large mesh of the first stent, the stent-in-stent technique can be achieved.

FIGS. 4A and 4B illustrate patterns of intertwined sections 8a.

Each intertwined section 8a has a first pattern shown in FIG. 4A or a second pattern shown in FIG. 4B. In the first pattern, the part of the winding 5a serving as the hill-shaped section 6 extends in the winding direction of the wire 5 from the radially outer side toward the radially inner side relative to the part of the winding 5a serving as the valley-shaped section 7. In the second pattern, the part of the winding 5a serving as the hill-shaped section 6 extends in the winding direction of the wire 5 from the radially inner side toward the radially outer side relative to the part of the winding 5a serving as the valley-shaped section 7. The winding direction of the wire 5 extends from the proximal end 2 toward the distal end 3 and extends from left to right in FIGS. 4A and 4B.

In FIG. 2A, only the intertwined sections 8a of the first pattern are arranged. FIG. 2B illustrates a mixture of the intertwined sections 8a of the first pattern and the intertwined sections 8a of the second pattern, such that the intertwined sections 8a of the first pattern and the intertwined sections 8a of the second pattern are alternately arranged.

As shown in FIG. 2A, the intertwined sections 8a in the intermediate part 4 may include the intertwined sections 8a of the first pattern alone, or may include the intertwined sections 8a of the second pattern alone. In this case, since the weaving method is the same from the proximal end to the distal end of the intermediate part 4, the stent 1 can be readily manufactured, and the manufacturing cost can be reduced.

As shown in FIG. 2B, the intertwined sections 8a in the intermediate part 4 may include both the intertwined sections 8a of the first pattern and the intertwined sections 8a of the second pattern. When the stent 1 moves in the longitudinal direction within a sheath of the delivery device during a releasing or retracting process of the stent 1, the stent 1 may receive a rotational force acting around the longitudinal axis A due to friction between radially outer parts of the wire 5 at the intertwined sections 8a and the inner surface of the sheath. If the intertwined sections 8a are of the first pattern alone or the second pattern alone, the rotational force may act only in one direction, possibly causing the stent 1 to become twisted. If the stent 1 includes the intertwined sections 8a of both the first pattern and the second pattern, the rotational force acts in both directions, so that the stent 1 can be prevented from being twisted. In particular, if the number of intertwined sections 8a of the first pattern and the number of intertwined sections 8a of the second pattern are the same, the stent 1 can be reliably prevented from being twisted.

FIGS. 5A and 5B illustrate patterns of the non-intertwined sections 8b.

Each non-intertwined section 8b has a first pattern shown in FIG. 5A or a second pattern shown in FIG. 5B. In the first pattern, the hill-shaped section 6 is located radially outward of the valley-shaped section 7. In the second pattern, the hill-shaped section 6 is located radially inward of the valley-shaped section 7.

In a case where the intermediate part 4 includes the non-intertwined sections 8b, the non-intertwined sections 8b in the intermediate part 4 may include the non-intertwined sections 8b of the first pattern alone, or may include the non-intertwined sections 8b of the second pattern alone. Alternatively, the non-intertwined sections 8b in the intermediate part 4 may include the non-intertwined sections 8b of both the first pattern and the second pattern.

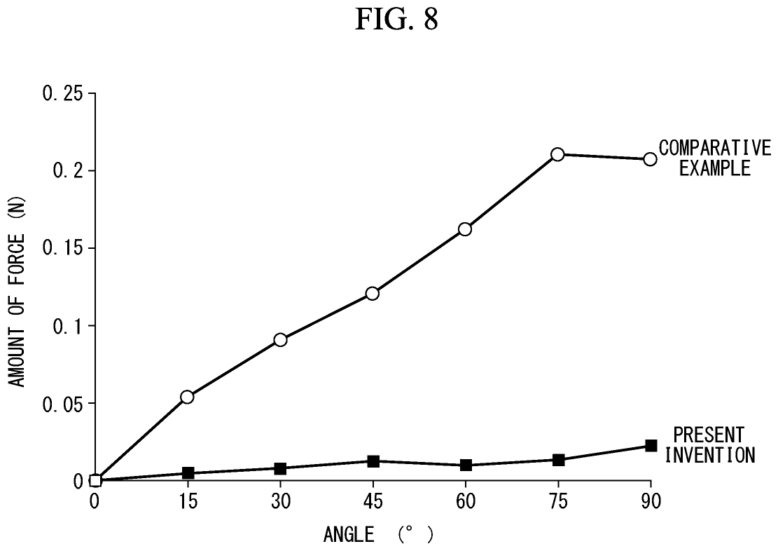

FIG. 8 is a graph illustrating measurement results of an amount of force required for bending the stent 1. In the graph in FIG. 8, the abscissa axis indicates the bending angle of the stent 1, and the ordinate axis indicates the amount of force.

In the case of a stent in the related art according to a comparative example, the amount of force increases with increasing bending angle. In contrast, in the case of the stent 1 according to the present invention, the amount of force hardly increases regardless of an increase in the bending angle. The amount of force at 90° is about one-tenth of the amount of force at 90° in the comparative example.

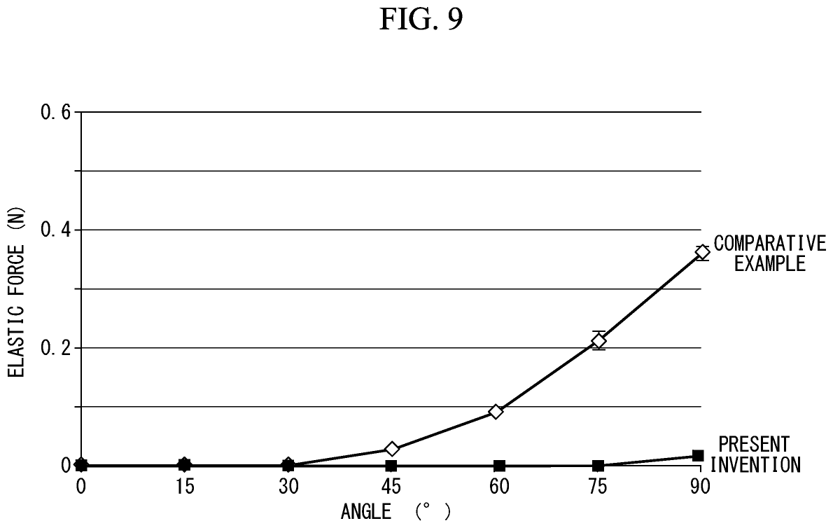

FIG. 9 is a graph illustrating measurement results of an elastic force of the stent 1 when bent. In the graph in FIG. 9, the abscissa axis indicates the bending angle of the stent 1, and the ordinate axis indicates the elastic force.

In the case of the stent in the related art according to the comparative example, the elastic force is not generated until 30°, but the elastic force increases with increasing bending angle in a range above 30°. In contrast, in the case of the stent 1 according to the present invention, the elastic force hardly occurs regardless of an increase in the bending angle. The elastic force at 90° is about one-tenth of the elastic force at 90° in the comparative example.

Accordingly, as compared with the stent according to the comparative example, it is clear that the stent 1 according to this embodiment can be bent to a large bending angle with an extremely small amount of force, and hardly generates an elastic force in the bent state.

In the measurements in FIGS. 8 and 9, the stent 1 according to the present invention has 12.5 hill-shaped sections, a pitch of 2 mm, a diameter of 8 mm, and an overall length of 80 mm, and includes the intertwined sections 8a for all the intersections 8. In the stent according to the comparative example, the four apexes of each mesh include intersection points where linear segments of a wire intersect with each other, as in the stent described in the Publication of Japanese Patent No. 4451421.

Next, an example of a method for manufacturing the stent 1 will be described.

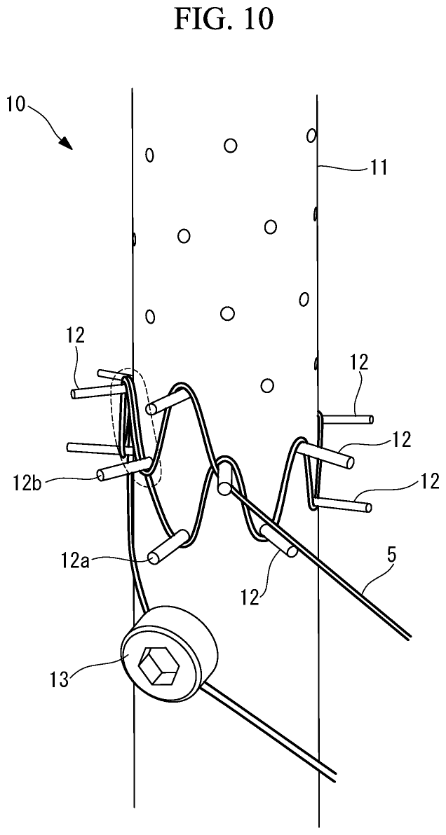

As shown in FIG. 10, the method for manufacturing the stent 1 includes a preparation step for preparing a jig 10 having a cylindrical shaft 11 and a weaving step for helically winding at least one wire 5 around the longitudinal axis of the shaft 11 from the proximal end toward the distal end of the shaft 11.

The jig 10 has a plurality of pins 12 attached to the outer peripheral surface of the shaft 11 and has holes for inserting the pins 12 therein at transition points on the outer peripheral surface of the shaft 11. The transition points on the shaft 11 correspond to the transition points in the development view in FIG. 3 and are intersection points between a plurality of circumference division lines and a plurality of length division lines. The circumference division lines extend in the longitudinal direction of the shaft 11 and equally divide the circumference of the shaft 11 into a plurality of segments. The length division lines extend in the circumferential direction of the shaft 11 and equally divide the length of the shaft 11 into a plurality of segments. In the preparation step, the pins 12 are attached to the corresponding transition points on the shaft 11. The plurality of pins 12 attached to the transition points are arranged along a helical path extending around the longitudinal axis of the shaft 11.

Subsequently, in the weaving step, one end of the wire 5 is fixed to an anchor pin 13, and the wire 5 is extended from the anchor pin 13 to a pin 12a serving as a starting point on the most-proximal length division line. Then, the wire 5 is extended in the circumferential direction of the shaft 11 from the starting point and is wound multiple times around the longitudinal axis of the shaft 11. Accordingly, a plurality of windings 5a are formed. In this case, the wire 5 is extended in the circumferential direction in a zigzag fashion while being routed alternately between pins 12 on one length division line and pins 12 on another length division line adjacent to the distal side of the one length division line. Accordingly, valley-shaped sections 7 are formed around the pins 12 on the one length division line, while hill-shaped sections 6 are formed around the pins 12 on the other length division line.

From the second turn onward, intertwined sections 8a are formed by intertwining the wire 5 with the wire 5 itself at one or more pins 12 on the one length division line. In detail, at each pin 12 on the one length division line, the wire 5 is routed over or under a part of the winding 5a serving as the hill-shaped section 6 from the radially outer side toward the radially inner side or from the radially inner side toward the radially outer side.

After the wire 5 is wound to the pin 12 serving as a finishing point on the most-distal length division line, the opposite ends of the wire 5 extending from the starting point to the finishing point are cut. Then, the proximal end of the wire 5 is joined to another part of the wire 5 near the starting point, and the distal end of the wire 5 is joined to another part of the wire 5 near the finishing point. Subsequently, the pins 12 are removed from the shaft 11, and the stent 1 is separated from the shaft 11.

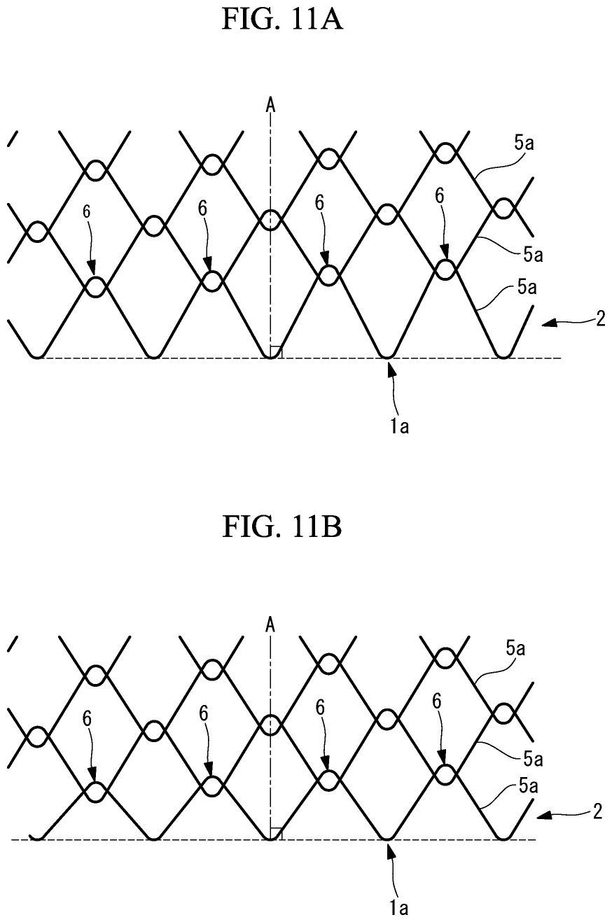

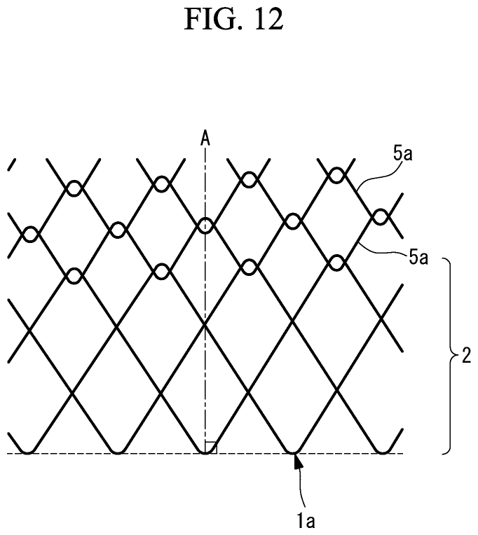

If the wire 5 is helically wound at the even pitch P1 over the entire length of the stent 1, opposite end surfaces 1a and 1b of the stent 1 are slanted relative to the longitudinal axis A, as shown in FIGS. 1 and 3. In this embodiment, at least one of the proximal end surface 1a and the distal end surface 1b may be perpendicular to the longitudinal axis A, as shown in FIGS. 11A to 12.

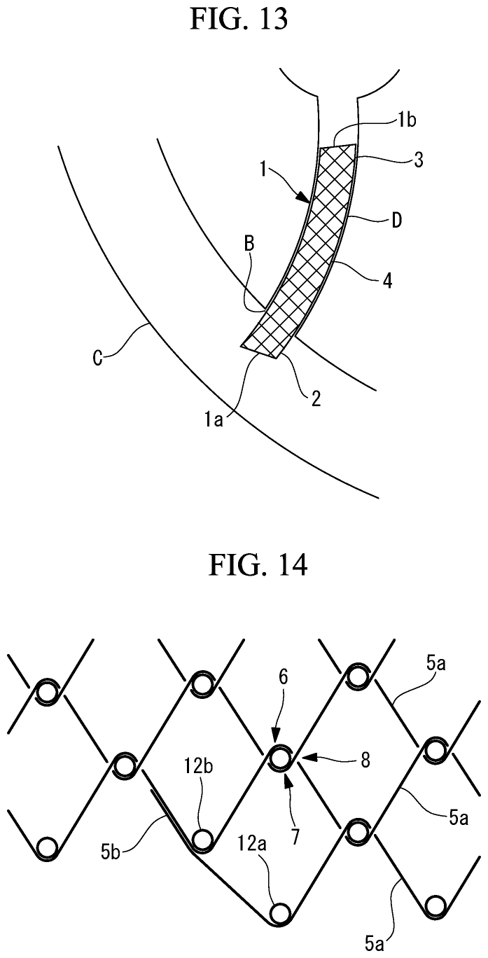

FIG. 13 illustrates an application example of the stent 1. As shown in FIG. 13, a commonly-known technique involves retaining the stent 1 in a lower bile duct D in a state where the proximal end 2 of the stent 1 protrudes from a papilla B to a duodenum C.

In this application example, the slanted end surfaces 1a and 1b may possibly have an effect on surrounding tissue. For example, the slanted proximal end surface 1a may possibly have an effect on the inner wall of the duodenum C, and the slanted distal end surface 1b may possibly become caught at the bifurcation area between the bile duct D and the cystic duct. Moreover, the slanted proximal end surface 1a may cause the stent 1 to be unstable at the papilla B, possibly resulting in aberration of the proximal end 2 within the bile duct D. In this application example, if the end surfaces 1a and 1b are perpendicular to the longitudinal axis A, the stent 1 can be made positionally stable, and the effect on the tissue can be reduced. Furthermore, if the distal end surface 1b is perpendicular to the longitudinal axis A, the retaining position of the stent 1 can be readily determined.

The perpendicular end surfaces 1a and 1b can be achieved by adjusting the manufacturing method. In FIG. 11A, the proximal end surface 1a perpendicular to the longitudinal axis A is formed by gradually increasing the height between the hill-shaped sections 6 and the valley-shaped sections 7 of the most-proximal winding 5a in a stepwise fashion in the circumferential direction, as compared with the height between the hill-shaped sections 6 and the valley-shaped sections 7 of other windings 5a. In FIG. 11B, the proximal end surface 1a perpendicular to the longitudinal axis A is formed by gradually decreasing the height between the hill-shaped sections 6 and the valley-shaped sections 7 of the most-proximal winding 5a in a stepwise fashion in the circumferential direction, as compared with the height between the hill-shaped sections 6 and the valley-shaped sections 7 of other windings 5a. The distal end surface 1b is also formed perpendicularly by using the same method as that for the proximal end surface 1a.

In the case of FIG. 11A, the most-proximal winding 5a at the proximal end 2 is longer in the direction along the longitudinal axis A, so that the expansion force of the proximal end 2 decreases. In the case of FIG. 11B, such a decrease in the expansion force can be prevented.

As an alternative to this embodiment in which the stent 1 has a rhombic-mesh pattern having the intersections 8 over the entire length, at least one of the proximal end 2 and the distal end 3 may be woven using another technique, as shown in FIG. 12.

FIG. 12 illustrates the proximal end 2 formed by braiding. The distal end 3 may also be formed by braiding. By using braiding, the perpendicular end surfaces 1a and 1b can be readily achieved.

In this embodiment, an end treatment for joining each end of the wire 5 to another part of the wire 5 may be performed in accordance with an arbitrary method. As shown in FIG. 14, in one example, an end of the wire 5 is extended from the pin 12a serving as the starting point to a final pin 12b on the most-proximal length division line, and is joined to a linear part of the wire 5. A dashed circle in FIG. 10 indicates a joint area of the end of the wire 5.

In the end treatment, it is desirable that high joint strength be obtainable between an end 5b and another part of the wire 5 and that the joint area do not interfere with the tissue and the delivery device. From this perspective, it is preferable that the end treatment be laser welding. Alternatively, the end treatment may be caulking or tight-winding that involves tightly winding the end to another part. Caulking and tight-winding are advantageous in terms of a simple process and low cost.

As an alternative to this embodiment in which the stent 1 is formed of a single wire 5 alone, the stent 1 may be formed of two or more wires 5. In this case, multiple wires 5 are woven in the form of a double start spring or a triple start spring, so that a stent 1 formed of a plurality of wires 5 can be manufactured.

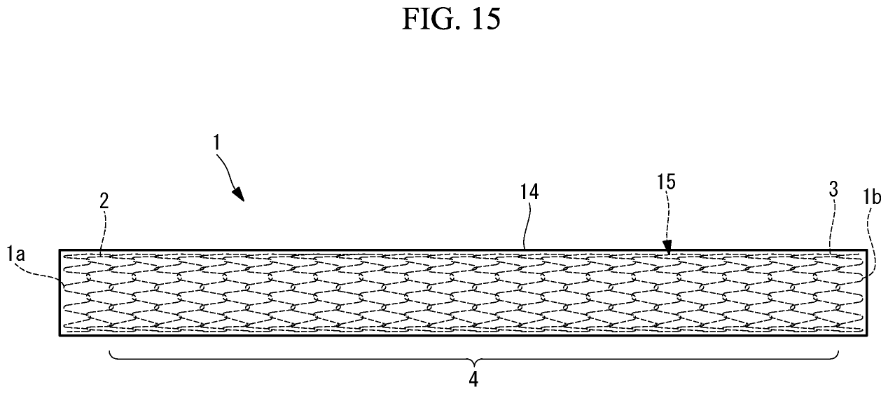

As shown in FIG. 15, in this embodiment, the stent 1 may further include a tubular cover 14 for preventing tumor invasion into the mesh or penetration of the mesh into the tissue. The cover 14 is disposed on at least the inner side and the outer side of a stent body 15 constituted of the proximal end 2, the distal end 3, and the intermediate part 4, and covers at least the inner side and the outer side of the stent body 15. In the example in FIG. 15, the cover 14 is disposed at the outer side of the stent body 15. The cover 14 is composed of any generic medical-grade material, such as PTFE (polytetrafluoroethylene) or silicone. The cover 14 may cover the entire length of the stent body 15 or may cover only a part of the stent body 15 in the lengthwise direction.

The following aspects can be also derived from the embodiments.

An aspect of the present invention provides a tubular stent formed of at least one wire. The stent includes a proximal end, a distal end, and an intermediate part interposed between the proximal end and the distal end. The intermediate part includes a plurality of annular windings extending in a circumferential direction around a longitudinal axis of the stent and arranged in a direction along the longitudinal axis. Each of the plurality of windings has a zigzag shape with hill-shaped sections and valley-shaped sections alternately arranged in the circumferential direction. Each of the hill-shaped sections is a part of each of the plurality of windings bent toward the proximal end. Each of the valley-shaped sections is a part of each of the plurality of windings bent toward the distal end. At least one of the hill-shaped sections included in each of the plurality of windings is intertwined with one of the valley-shaped sections of another neighboring one of the plurality of windings to form an intertwined section.

According to this aspect, each winding in the intermediate part has at least one hill-shaped section that forms an intertwined section together with a valley-shaped section of another neighboring winding. Specifically, each winding in the intermediate part is intertwined with another neighboring winding via at least one hill-shaped section. Each intertwined section functions as a coupler that couples two neighboring windings to each other. Therefore, the tubular shape of the stent can be maintained with the wire alone.

Furthermore, the hill-shaped section and the valley-shaped section at each intertwined section are shiftable relative to each other in accordance with a bending force applied to the stent. Specifically, when the stent bends, deformation hardly occurs or does not occur at the parts of the two windings serving as the hill-shaped section and the valley-shaped section at each intertwined section, so that an elastic force that restores the bent stent to a linear shape hardly occurs or does not occur at the intertwined section. Therefore, a stent that has increased flexibility and that is bendable with a small force can be achieved.

In the above aspect, each of the plurality of windings may be coupled to the another neighboring one of the plurality of windings at the intertwined section alone.

According to this configuration, the flexibility of the stent is further increased, thereby achieving a stent that hardly generates or does not generate an elastic force even at a large bending angle.

In the above aspect, all the hill-shaped sections and all the valley-shaped sections in the intermediate part may form intersections, and the hill-shaped sections of one of the plurality of windings and the valley-shaped sections of another one of the plurality of windings adjacent to the one of the plurality of windings may respectively overlap each other at the intersections in a radial direction of the stent.

According to this configuration, the intermediate part has a uniform structure over the entire length of the intermediate part, so that the stent can be manufactured more readily.

In the above aspect, the intersections may be helically arranged around the longitudinal axis. Furthermore, the at least one wire may include a single wire, and the stent may be formed of the single wire that is helically wound around the longitudinal axis.

According to this configuration, the stent can be manufactured using a simpler method.

In the above aspect, at least one of the intersections may be the intertwined section, and each of the remaining intersections may be non-intertwined section where the hill-shaped sections and the valley-shaped sections are arranged parallel to each other in the radial direction without being intertwined with each other.

Because the hill-shaped sections and the valley-shaped sections at the non-intertwined sections are not restrained by each other, the hill-shaped sections and the valley-shaped sections are freely shiftable in accordance with a bending force applied to the stent. Specifically, when the stent bends, deformation does not occur at the parts of two windings serving as the hill-shaped section and the valley-shaped section at each non-intertwined section, and an elastic force that restores the bent stent to a linear shape does not occur at the non-intertwined section. Therefore, with one or more of the intersections being non-intertwined sections, the flexibility of the stent can be further increased.

In the above aspect, the intermediate part may include either one of the non-intertwined section of a first pattern and the non-intertwined section of a second pattern. Alternatively, the intermediate part may include both the non-intertwined section of a first pattern and the non-intertwined section of a second pattern. The first pattern is a pattern in which each of the hill-shaped sections is located outward of each of the corresponding valley-shaped sections in the radial direction. The second pattern is a pattern in which each of the hill-shaped sections is located inward of each of the corresponding valley-shaped sections in the radial direction.

According to this configuration, the patterns of the non-intertwined sections can be selected as appropriate.

In the above aspect, the intermediate part may include either one of the intertwined section of a first pattern and the intertwined section of a second pattern. The first pattern is a pattern in which a part of the winding serving as each of the hill-shaped sections extends in a winding direction of the wire from a radially outer side toward a radially inner side of the stent relative to a part of the winding serving as each of the corresponding valley-shaped sections. The winding direction of the wire extends from the proximal end toward the distal end. The second pattern is a pattern in which the part of the winding serving as each of the hill-shaped sections extends in the winding direction from the radially inner side toward the radially outer side relative to the part of the winding serving as each of the corresponding valley-shaped sections.

According to this configuration, since the intertwined sections in the intermediate part all have the same pattern, the stent can be readily manufactured, and the manufacturing cost of the stent can be reduced.

In the above aspect, the intermediate part may include both the intertwined section of a first pattern and the intertwined section of a second pattern.

As a solution for delivering the stent to a narrow area, a delivery device having a sheath that accommodates the stent is used. The stent may sometimes receive a twisting force acting around the longitudinal axis of the stent when a radially outer part of a winding at any of the intertwined sections and the inner surface of the sheath come into contact with each other. With the mixture of the first pattern and the second pattern, twisting forces occur in opposite directions from each other so as to cancel each other out. Accordingly, the stent can be prevented from being twisted.

In the above aspect, at least one of ends of the wire may be joined to another part of the wire by welding, caulking, or tight-winding.

According to this configuration, the end of the wire can be readily joined to another part thereof with sufficient strength.

In the above aspect, the stent may further include a tubular cover disposed on at least an inner side and an outer side of a stent body including the proximal end, the distal end, and the intermediate part.

Another aspect of the present invention provides a stent delivery system including a delivery catheter that includes a tubular outer cylindrical unit and an inner cylindrical unit inserted in the outer cylindrical unit, and the aforementioned stent fitted within a distal end of the delivery catheter. The delivery catheter retains the stent such that the stent is releasable in accordance with relative movement between the outer cylindrical unit and the inner cylindrical unit in a longitudinal direction of the outer cylindrical unit.

Another aspect of the present invention provides a stent manufacturing method including a step for preparing a jig that includes a cylindrical shaft and a plurality of pins attached to an outer peripheral surface of the shaft, and a step for helically winding at least one wire around a longitudinal axis of the shaft from a proximal end toward a distal end of the shaft. The step for preparing the jig includes a step for attaching the pins to a plurality of transition points on the outer peripheral surface of the shaft. The transition points are intersection points between a plurality of circumference division lines and a plurality of length division lines. The plurality of circumference division lines extend in a longitudinal direction of the shaft and divide a circumference of the shaft into a plurality of segments. The plurality of length division lines extend in a circumferential direction of the shaft and divide a length of the shaft into a plurality of segments. The step for helically winding the wire includes a step for extending the at least one wire in the circumferential direction in a zigzag fashion while routing the at least one wire alternately between the pins on one of the length division lines and the pins on another one of the length division lines adjacent to a distal side of the one of the length division lines. The step for extending the at least one wire in the zigzag fashion includes a step for forming an intertwined section by intertwining the wire with the wire itself using at least one of the pins on the one of the length division lines.

### REFERENCE SIGNS LIST

1 stent
1a, 1b end surface
2 proximal end
3 distal end
4 intermediate part
5 wire
5a winding
6 hill-shaped section
7 valley-shaped section
8 intersection
8a intertwined section
8b non-intertwined section
10 jig
11 shaft
12 pin
20 delivery catheter
100 stent delivery system

1. A tubular stent formed of at least one wire, the stent comprising:
a proximal end, a distal end, and an intermediate part interposed between the proximal end and the distal end,
wherein the intermediate part includes a plurality of annular windings extending in a circumferential direction around a longitudinal axis of the stent and arranged in a direction along the longitudinal axis,
wherein each of the plurality of windings has a zigzag shape with hill-shaped sections and valley-shaped sections alternately arranged in the circumferential direction, each of the hill-shaped sections being a part of each of the plurality of windings bent toward the proximal end, each of the valley-shaped sections being a part of each of the plurality windings bent toward the distal end,
wherein all the hill-shaped sections and all the valley-shaped sections form intersections, the hill-shaped sections of one of the plurality of windings and the valley-shaped sections of another one of the plurality of windings adjacent to the one of the plurality of windings respectively overlapping each other at the intersections in a radial direction of the stent,
wherein the intersections are helically arranged around the longitudinal axis, and
wherein distances in the direction of the longitudinal axis between the hill-shaped sections and the valley-shaped sections of a first winding of the plurality of windings are different from each other in a circumferential direction of the stent, the hill-shaped sections and the valley-shaped sections of the first winding forming at least one of the proximal end and the distal end.

2. The stent according to claim 1, wherein the distances in the direction of the longitudinal axis between the hill-shaped sections and the valley-shaped sections of the first winding are gradually changed in the circumferential direction of the stent.

3. The stent according to claim 1, wherein a distance in the direction of the longitudinal axis between the hill-shaped sections and the valley-shaped sections of a second winding of the plurality of windings is different from a distance in the direction of the longitudinal axis between the hill-shaped sections and the valley-shaped sections of the first winding, the second winding being adjacent to the first winding.

4. The stent according to claim 1, wherein a distance in the direction of the longitudinal axis between the hill-shaped sections and the valley-shaped sections of a second winding of the plurality of windings is larger than a distance in the direction of the longitudinal axis between the hill-shaped sections and the valley-shaped sections of the first winding, the second winding being adjacent to the first winding.

5. The stent according to claim 1, wherein a distance in the direction of the longitudinal axis between the hill-shaped sections and the valley-shaped sections of a second winding of the plurality of windings is smaller than a distance in the direction of the longitudinal axis between the hill-shaped sections and the valley-shaped sections of the first winding, the second winding being adjacent to the first winding.
